(19) United States
(12) Patent Application Publication
(10) Pub. No.: US 2025/0339646 A1
(43) Pub. Date: Nov. 6, 2025

(54) WATER RESISTANT CATHETER COVER AND SECUREMENT SYSTEM

(71) Applicant: Cath Dry Global Corp., Los Angeles, CA (US)

(72) Inventor: Pascal Dabel, Los Angeles, CA (US)

(21) Appl. No.: 18/868,215

(22) PCT Filed: May 25, 2022
(86) PCT No.: PCT/US2022/030883

§ 371 Date: Nov. 22, 2024

(51) Int. Cl. A61M 25/02

(52) U.S. Cl. CPC A61M 25/02; A61M 2025/0253

(57) ABSTRACT

A catheter cover securement device and a method of using the same with a catheter cover sleeve. The catheter cover securement device has an anchoring body with a generally flat top surface, wherein the top surface has a longitudinal length substantially equal to a sleeve width of the sleeve. A nesting pocket is centrally recessed in the anchoring body so that a catheter hub may be received in the nesting pocket through a void in the top surface. A retaining strap may be pivotably connected to the anchoring body so as to be movable between an open position and a closed position securing the nested catheter hub and sandwiching a portion of the sleeve in a flat condition between the retaining strap and the flat top surface.

### BACKGROUND OF THE INVENTION

The present invention relates to a catheter cover and, more particularly, to a method of improving catheter covers that sterilely protect the catheter from water. The method embodies a catheter cover securement device that securely anchors a catheter to a patient's skin while at the same time not damaging or disturbing the catheter cover. As a result, the catheter cover securement device may be non-sterile, and as such may be made from a wider range of materials and thereby more affordable relative to prior art sterile catheter securement devices.

Dialysis patients that must use catheters are not allowed to shower or swim for weeks and up to months at a time. The patients thereby become dirty, uncomfortable, and are more prone to infection. The only way a patient may clean themselves is by using a washcloth or sponge which promotes cross contamination of the catheter site. Further, the patients are at increased risk for blood stream infections since there are no appropriate methods of protecting the catheter site.

Prior to the inventor's U.S. Pat. No. 10,137,292 patent, methods of covering the catheter, relied on gauze-based solutions. However, the gauze bases only protected the entry site into the skin and are not water resistant. The gauze did not prevent contaminants from contacting the catheter or entry site into the skin. The gauze and tape absorbed moisture, dead skin cells, and bacteria. All of the above promotes infection instead of preventing it. Further, the gauze and tape fall off easily leaving little to no protection.

As can be seen, there is a need for a sterile cover that protects the catheter from the elements and allows for an improvement in quality of life due to increased hygiene.

Furthermore, it is known in the art relating to catheters that after insertion of a catheter through a patient's skin into a vein, the catheter is ideally secured to prevent the catheter from slipping out of the patient's vein, which can happen when the catheter and its hub flop around when the patient is mobile. Commonly, a catheter securement device is used to hold the catheter hub to the patient's skin. However, when the catheter hub is already sheathed in the sterile cover of the present invention, conventional catheter securement devices would damage or disturb the sterile cover, frustrating the sterile cover's purpose. Therefore, a need exists for an anchoring solution that can secure a catheter hub sheathed in a sterile catheter cover contemplated herein.

The present catheter securement device is designed to be used to anchor catheter hubs sheathed in a water-resistant catheter cover. For example, one intended use of the present catheter cover securement device is to secure PICCs (Peripherally Inserted Central Catheters) sheathed in the water-resistant catheter cover. The present catheter cover securement device prevents stresses applied to the catheter hub or fittings thereof from transferring to the catheter insertion site and causing catheter migration or removal as well as insertion site irritation.

### SUMMARY OF THE INVENTION

In one aspect of the present invention, a system for securing a catheter, wherein the catheter is sheathed in a catheter covering device, the system includes the following: a water-resistant sleeve having a sleeve width, wherein the water-resistant sleeve sheathes the catheter; and a catheter cover securement device providing an anchoring body configured to be mounted on a patient; a flat top surface of the anchoring body, wherein a longitudinal length of the flat top surface is equal to or greater than the sleeve width; and a pocket recessed in the anchoring body, wherein a hub of the catheter is received in the pocket so that an engaged portion of the water-resistant sleeve engages the flat top surface in a flat condition, wherein the pocket communicates via a void with the flat top surface, wherein the flat top surface is continuous along said longitudinal length except for said void; further including a retaining strap movable relative to the flat top surface in such a way as to move between an open position and a closed position sandwiching said engaged portion against the flat top surface, and wherein the water-resistant sleeve further comprises a rim forming an opening into the sleeve; and wherein the catheter cover securement device further includes a base member having an adhesive side and a non-adhesive side, wherein the anchoring body is mounted on the non-adhesive side, and wherein a curved upper edge of the base member has a radius of curvature generally concentric with a radius of the rim.

In another aspect of the present invention, a method of securing a catheter to a patient with a non-sterile securement device, the method includes the following: sheathing the catheter in a water-resistant sleeve; adhering the non-sterile securement device to the patient so that a hub of the catheter is secured in the non-sterile securement device and a portion of the sleeve engaged by the non-sterile securement device is in a flat condition on opposing ends of its respective sleeve width, wherein the non-sterile securement device is disposed adjacent a rear surface of the sleeve, wherein the non-sterile securement device provides a recessed pocket; nesting a portion of the catheter in the recessed pocket; and sandwiching a portion of the rear surface and an opposing front surface of the sleeve in a flat condition to the non-sterile securement device by way of a retaining strap operatively associated with the non-sterile securement device.

In yet another aspect of the present invention, a method of improving a catheter cover attached to a patient, wherein the catheter includes a water-resistant sleeve sheathing a catheter hub, wherein the sleeve has a sleeve width, the method providing a catheter cover securement device having a base member with an adhesive side and a non-adhesive side; an anchoring body mounted on the non-adhesive side; a flat top surface of the anchoring body, wherein a longitudinal length of the flat top surface is equal to or greater than the sleeve width; a pocket recessed in the anchoring body; and a retaining strap movable relative to the flat top surface in such a way as to move between an open position and a closed position; adhering the adhesive side to the patient so that the catheter cover securement device is between the patient and the sleeve, wherein the catheter hub is adjacent the pocket; urging the catheter hub into the pocket; and moving the retaining strap to the closed position so that the retaining strap sandwiches an engaged portion of the sleeve in a flat condition against the flat top surface.

In another embodiment of the present invention, a kit embodies the above-mentioned system and methods, wherein the kit includes a set of instructions for using the present invention, wherein the set of instructions instruct mounting the anchoring body on the patient rearward of the water-resistant sleeve in such a way that the retaining strap can sandwich the water resistant sleeve against the flat top surface, and wherein the set of instructions may be printed.

These and other features, aspects and advantages of the present invention will become better understood with reference to the following drawings, description and claims.

### BRIEF DESCRIPTION OF THE DRAWINGS

FIG. 1 is a front elevation view of a catheter cover, shown in use, prior to utilization of an exemplary embodiment of a catheter cover securement device of the present invention.

FIG. 2 is a rear elevation view of an exemplary embodiment of the catheter cover securement device of the present invention.

FIG. 3 is a front elevation view of an exemplary embodiment of the catheter cover securement device of the present invention.

FIG. 4 is a front elevation view of an exemplary embodiment of the catheter cover securement device of the present invention.

FIG. 5 is a front elevation view of an exemplary embodiment of the catheter cover securement device of the present invention.

FIG. 6 is a front elevation view of an exemplary embodiment of the catheter cover securement device of the present invention.

FIG. 7 is a front elevation view of an exemplary embodiment of the catheter cover securement device of the present invention, shown in an open condition.

FIG. 8 is a front elevation view of an exemplary embodiment of the catheter cover securement device of the present invention, shown in a closed condition.

FIG. 9 is a section view of FIG. 1, wherein the tab 70 and the first adhesive strip 72 are engaged.

### DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims.

The present invention includes a plastic covering shield for hemodialysis catheters to prevent infections and water exposure. The present invention covers the entire hemodialysis catheter and entry site into the skin, therefore allowing patients to freely shower and swim since the present invention is water resistant and impermeable. The closed-circuit environment of the present invention also substantially limits contamination and bacterial, viral or fungal exposure to the catheter/skin entry site (i.e., open wound), and therefore reduces the number of infections. The present invention may not contain gauze and may include polyurethane sleeve which allows patients to shower and swim due to the water-resistant properties. The entire catheter unit and skin entry site is protected which decreases pathogen exposure.

The polyurethane sleeve has a high moisture/water vapor transmission rate which will prevent accumulation of moisture (sweat and condensation) inside of the closed-circuit environment once applied to the skin. Water cannot get into the device, but moisture freely passes out at a rate higher than that of sweat produced from the area of skin being protected.

Broadly, an embodiment of the present invention provides a catheter covering device with at least a sleeve and an adhesive film. The catheter covering device may further include a water indicator attached to the adhesive film and a desiccant paper attached to the water indicator. The adhesive film may include a larger diameter than the water indicator and the desiccant paper. Thereby the adhesive film is exposed around the circumference of the water indicator and desiccant paper. A catheter may be inserted into the sleeve and the adhesive film may be adhered to a user's skin. The catheter covering device may thereby protect the catheter from the elements. If moisture enters the catheter covering device, the desiccant paper may absorb the moisture and activate the water indicator, indicating to the user that the catheter covering device has been contaminated. The white to red color change is visible through the device.

More particularly, a catheter cover securement device in accordance with the present invention includes a flexible base member having an adhesive side and an opposite non-adhesive side. The catheter cover securement device has an anchoring body with a generally flat top surface, wherein the top surface has a longitudinal length substantially equal to a sleeve width of the sleeve. A nesting pocket is centrally recessed in the anchoring body so that a catheter hub may be received in the nesting pocket through a void in the top surface. A retention strap may be pivotably connected to the anchoring body so as to be movable between an open position and a closed position securing the nested catheter hub and sandwiching a portion of the sleeve in a flat condition between the retaining strap and the flat top surface.

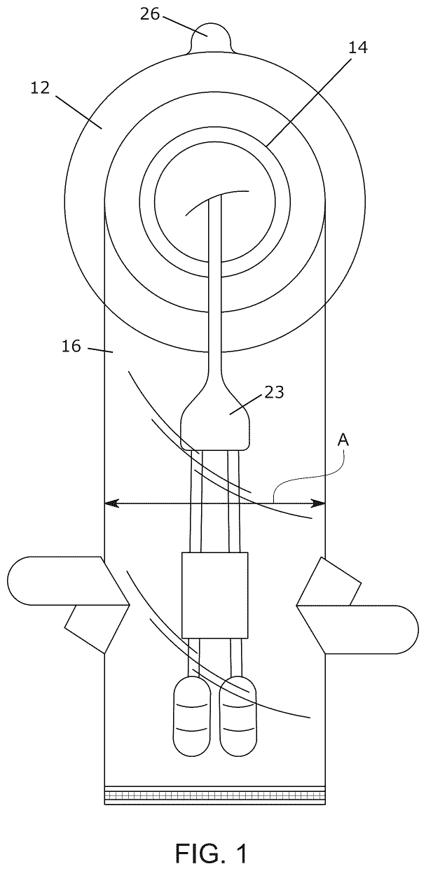

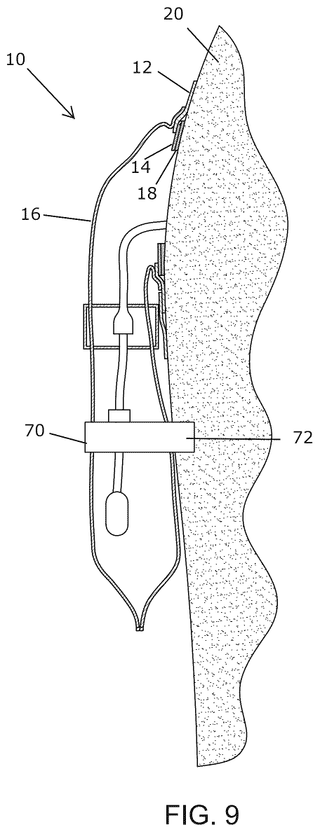

Referring to FIGS. 1 and 9, the present invention may include a catheter covering device 10. The catheter covering device 10 may include a water-resistant sleeve 16. The water-resistant sleeve 16 may include a first end and a second end. The first end may include a rim forming an opening into the sleeve 16. The second end may be a closed end. The water-resistant sleeve 16 may be made of a polymer, such as a plastic. For example, the water-resistant sleeve 16 may be made of an Ultraviolet protective polyethylene. In certain embodiments, the water-resistant sleeve 16 may be approximately seven and a half inches long with about a one-inch diameter. The sleeve 16 may be formed to fit over the catheter 22 of a user 20.

The present invention further includes an adhesive film 12. The adhesive film 12 is attached to the rim of the water-resistant sleeve 16. The adhesive film 12 includes an opening that leads into the sleeve 16. In certain embodiments, the adhesive film 12 includes a first side and a second side. The rim of the sleeve 16 may be sealed, such as radiofrequency welded to the first side of the adhesive film 12. The second side of the adhesive film 12 may include the adhesive material. The adhesive film 12 and the water-resistant sleeve 16 may prevent water from contacting the catheter 22 and the skin surrounding the catheter 22. In certain embodiments, the adhesive film 12 is a waterproof tape, such as 3M tegaderm®. The adhesive film 12 may have a round shape, however the waterproof tape 12 may be any shape desired.

In certain embodiments, the present invention may include a water indicator 14. The water indicator 14 may be attached to the adhesive film 12. The water indicator 14 may be attached circumferentially around the opening of the adhesive film 12. In certain embodiments, the water indicator 14 may change color, from white to red, when moisture comes into contact with the water indicator 14. This indicates to the user 20 that moisture has entered the barrier and the user 20 may exit the water and remove the sleeve 16 and replace with a new device. The water indicator 14 may include 3M 5557® water indicator tape, an indigo color indicating paper, an electronic tape detector or the like.

The present invention may further include a desiccant paper 18. The desiccant paper 18 may be attached to the water indicator 14. In certain embodiments, the desiccant paper 18 may include an opening aligning with the opening of the adhesive film 12 and the water indicator 14. In certain embodiments of the present invention, the water indicator 14 may be attached to the first side of the adhesive film 12 and the desiccant paper 18 may be attached to the opposite side of the water indicator 14. Therefore, the water indicator 14 may be sandwiched in between the adhesive film 12 and the desiccant paper 18. The desiccant paper 18 may absorb moisture and transmits the moisture to the water indicator 14, which may change color when exposed to water.

In certain embodiments, the present invention may further include additional components. For example, a tab 26 may extend from the perimeter of the adhesive film 12. The tab 26 may or may not include an adhesive material. Therefore, when the user 20 has the catheter covering device 10 attached to their skin, the user 20 may pull the tab 26 in order to remove the catheter covering device 10.

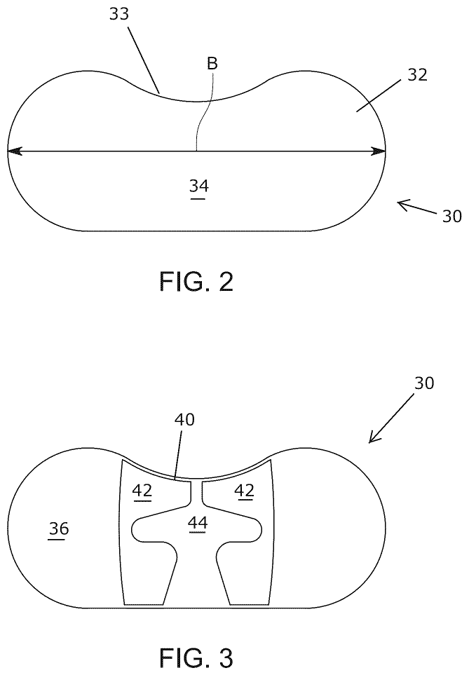

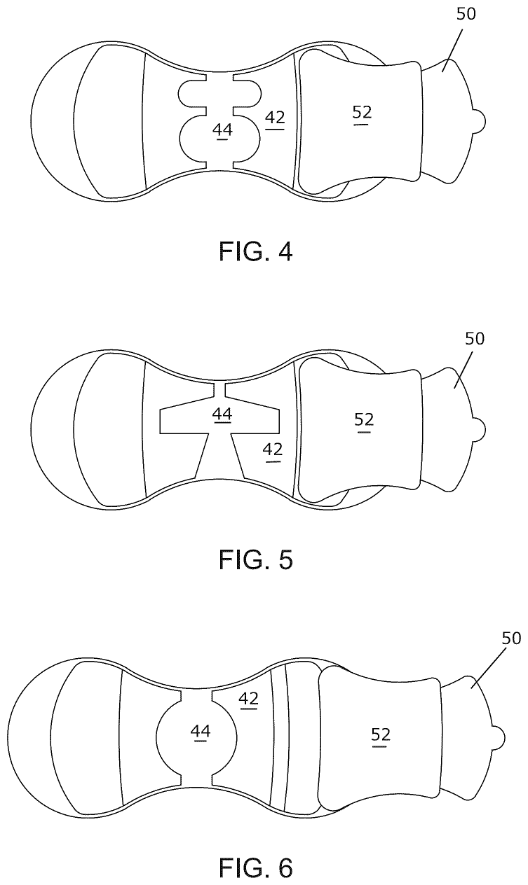

Referring to FIGS. 2 through 8, the present invention may include a catheter cover securement device 30 in accordance with the present invention. A bottom side of the securement device 30 can be adhered to a patient's skin 20 so that that an anchoring body 40 of the securement device 30 is disposed between the patient's skin and the water-resistant sleeve 16 that houses the catheter hub 23. The anchoring body 40 provides a flat top surface 42 and a nesting pocket 44. The nesting pocket 44 is dimensioned to urgedly engage the catheter hub 23 and a respective portion of the sleeve 16, while the top surface 42 supports an engaged portion the water-resistant sleeve 16 in a flat, undisturbed condition. A retaining strap 50 is moveable from an open position to a closed position covering the nesting pocket 44, thereby sandwiching the engaged portion of the water-resistant sleeve 16 against the top surface 42. The retaining strap 50 can be woven cotton, rayon fabric, woven material or the like that is coated with a suitable adhesive to form a locked engagement with the sleeve 16 and the portion of the base member 32 it engages in the closed position. Once the catheter hub 23 is secured by the securement device 30, stresses and forces applied in any direction on the fittings connected to the catheter 22 will not cause the catheter 22 to move and thereby irritate the insertion site.

Attached to the retaining strap 50 may be an engagement surface 52 that is dimensioned and adapted to be generally or approximately coextensive with the top surface 42. Accordingly, as a result of the retaining strap 50 being moved to the closed position, the engagement surface interfaces with the top surface, closing off access to the nesting pocket 44. In certain embodiments, the top surface 42 may be domed, convex, or the like, and the engagement surface 52 of the retaining strap 50 adapted to conform to the domed top surface 42.

The present invention may include a flexible base member 32 having an adhesive side 34 for adherence to a surface such as a patient's skin, and an opposite non-adhesive side 36. The surface of the adhesive side 34 includes an adhesive such as a medical grade adhesive or similar thereon. The base member 32 may be a generally flat, planar pad made of a foam, film, or the like.

The anchoring body 40 is mounted on the non-adhesive side 36 of the base member 32. The anchoring body 40 may be made of any suitable flexible, stretchable elastomeric material, or the like.

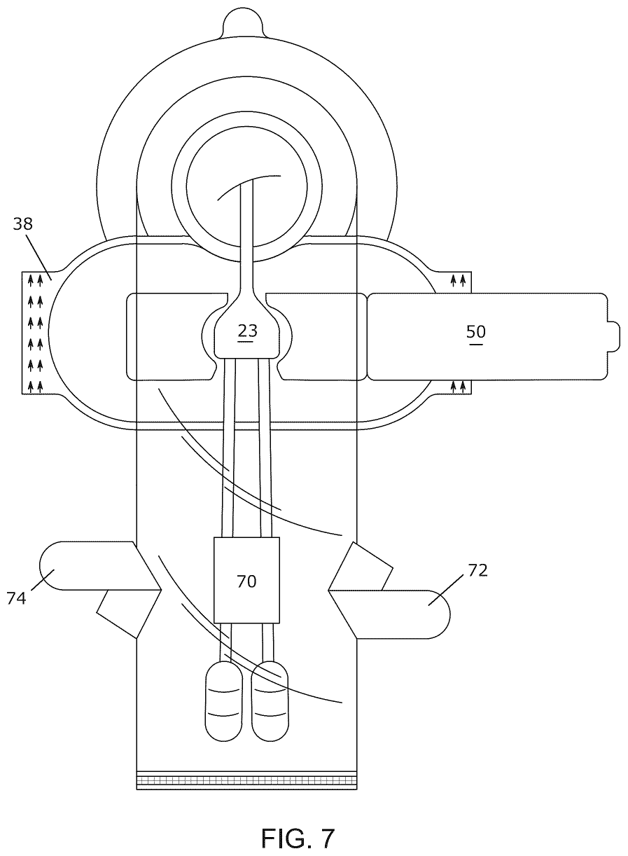

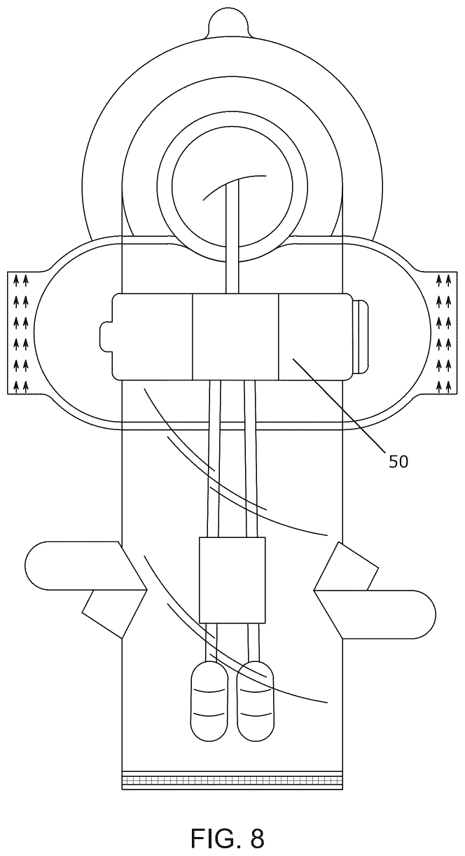

The anchoring body 40 or, in other embodiments, the base member 32 may have a retaining strap 50 moveably or pivotably connected thereto in such a way that the retaining strap 50 is movable between an open condition (FIG. 7) and a closed condition (FIG. 8). The retaining strap 50 may have a flat engagement surface 52 that is generally coextensive with the top flat surface 42 of the anchoring body 40. Further, the flat engagement and top surfaces 52 and 42 each have a longitudinal length equal to or greater than a sleeve width ‘A’ of the water-resistant sleeve 16 so that in the closed condition an engaged portion of the water-resistant sleeve 16 is sandwiched in a flat condition between surfaces 52 and 42. The longitudinal axis for the surfaces 52 and 42 as well as the base member 32 is in the direction of the base length ‘B’ of the base member 32, as illustrated in FIG. 2.

The nesting pocket 44 may be centrally disposed relative to the top surface 42. The anchoring body 40 may be generally symmetrical about a longitudinal axis and/or a transverse axis (i.e., x and y axes) of the securement device 30.

The base member 32 may have a curvature along an upper edge 33, wherein the curved edge 33 is dimensioned and adapted to have a radius approximately equal to or slightly greater than the radius of the circular water indicator 14. The curved edge 33 facilitates a hugging condition, wherein the curved edge 33 abuts a lower circumference of the circular water indicator 14, as illustrated in FIG. 7, thereby the curved edge 33 acts as a guide for users when properly adhering the securement device 30 to the patient's skin 20.

The securement device 30 may include a pull-away liner 38 contacting the adhesive side 34 of the base member 32 to prevent the securement device 30 from adhering to another object prior to its use (i.e., during storage).

Referring the FIG. 9, in some embodiments, a tab 70 is joined to the sleeve 16 proximate to the second end and on a side away from the human user. A first adhesive strip 72 is joined to the tab 70 and the sleeve 16 and configured to be attached to skin of a human user. A second adhesive strip 74 is joined to the tab 70 and the sleeve 16. The second adhesive strip 74 is linearly aligned with the tab 70 and the first adhesive strip 72 and is configured to be attached to the skin of the human user.

A method of using the catheter cover may include the following. First a user may provide the catheter covering device mentioned above. The user may groom an area of the body surrounding the catheter, which may include shaving and/or cleaning the area. The user may place the catheter through the opening and within the sleeve. The air may be squeezed out of the plastic sleeve. The rim of the water resistance sleeve may be adhered to the body by using waterproof tape around the catheter insertion site into the skin. The waterproof tape may be pressed firmly against dry and hairless skin around the catheter. This may prevent water from getting into the device or making contact with the catheter in the shower or pool. If water were to accidentally get through the waterproof tape, the water may come into contact with the water indicator which may cause a color change, for example from white to red, blue to white or the like. The patient may then know to get out of the shower or pool and dry themselves off thoroughly. The device may then be removed from the skin by pulling on the tab and discarded.

A method of making the catheter cover may include the following. Medical grade skin tape may be cut into circular shape. The color changing/water indicating tape may be cut with desiccant paper into a smaller circular shape, i.e. a smaller diameter. The present invention may be layered so that the water indicator is between the adhesive film and the desiccant paper. An opening may be cut into the middle or the circle making a ring. These components may be glued or taped together. Polyurethane plastic may then be shaped into a sleeve. The opening of the of the sleeve may be a similar size of the hole cut into the combined components above. The sleeve may be radiofrequency welded onto the non tape/skin side of the above unit. The entire device may need to be created in a sterile environment.

As used in this application, the term “about” or “approximately” refers to a range of values within plus or minus 10% of the specified number. And the term “substantially” refers to up to 90% or more of an entirety. Recitation of ranges of values herein are not intended to be limiting, referring instead individually to any and all values falling within the range, unless otherwise indicated, and each separate value within such a range is incorporated into the specification as if it were individually recited herein. The use of any and all examples, or exemplary language (“e.g.,” “such as,” or the like) provided herein, is intended merely to better illuminate the embodiments and does not pose a limitation on the scope of the embodiments or the claims. No language in the specification should be construed as indicating any unclaimed element as essential to the practice of the disclosed embodiments.

It should be understood, of course, that the foregoing relates to exemplary embodiments of the invention and that modifications may be made without departing from the spirit and scope of the invention as set forth in the following claims.

1.-12. (canceled)

13. A method of securing a catheter to a patient, wherein the catheter includes an exterior portion that extends outside of the patient, wherein the exterior portion includes a catheter hub, the method comprising:
(a) securing a rim of a water-resistant sleeve to skin of the patient, wherein the catheter extends through the rim;
(b) after securing the rim of the water-resistant sleeve to the skin of the patient, securing a catheter cover securement device to the skin; and
(c) after securing the rim of the water-resistant sleeve to the skin of the patient, securing the catheter hub of the catheter, which is surrounded by the water-resistant sleeve, within a three-dimensional pocket defined by a three-dimensional anchoring body of the catheter cover securement device.

14. The method of claim 13, further comprising positioning the three-dimensional anchoring body of the catheter cover securement device between the skin and the water-resistant sleeve that surrounds the catheter hub after securing the rim of the water-resistant sleeve to the skin of the patient.

15. The method of claim 13, further comprising surrounding the exterior portion of the catheter extending outside of the patient within the water-resistant sleeve prior to securing the rim of the water-resistant sleeve to the skin of the patient.

16. The method of claim 15, further comprising inserting the catheter into the patient such that the exterior portion of the catheter extends outside of the patient prior to surrounding the exterior portion of the catheter extending outside of the patient within the water-resistant sleeve.

17. The method of claim 13, wherein the act of securing the catheter cover securement device to the skin is performed before the act of securing the catheter hub of the catheter, which is surrounded by the water-resistant sleeve, within the three-dimensional pocket.

18. The method of claim 13, wherein the act of securing the catheter cover securement device to the skin is performed after the act of securing the catheter hub of the catheter, which is surrounded by the water-resistant sleeve, within the three-dimensional pocket.

19. The method of claim 13, the catheter cover securement device comprising a base member having an adhesive side and a non-adhesive side, and an anchoring body mounted on the non-adhesive side, the method further comprising adhering the adhesive side to the skin so that the catheter cover securement device is positioned between the skin and the water-resistant sleeve so that the catheter hub is adjacent the three-dimensional pocket.

20. The method of claim 13, further comprising after securing the catheter hub within the three-dimensional pocket, moving a retaining strap of the catheter cover securement device from an open configuration allowing for insertion or removal of the catheter hub from the three-dimensional anchoring body to a closed configuration inhibiting insertion or removal of the catheter hub from the three-dimensional anchoring body.

21. The method of claim 20, further comprising adhering the retaining strap to a base member of the catheter cover securement device that is secured to the skin in the closed configuration.

22. The method of claim 13, further comprising after securing the catheter hub within the three-dimensional pocket, moving a retaining strap of the catheter cover securement device from an open configuration allowing for insertion or removal of the catheter hub from the three-dimensional anchoring body to a closed configuration to conform an engagement surface of the retaining strap to a top surface of the three-dimensional anchoring body that defines the three-dimensional anchoring body.

23. The method of claim 13, wherein the three-dimensional anchoring body includes first and second non-contiguous projections defining the three-dimensional pocket, wherein the act of securing the catheter hub of the catheter within the three-dimensional pocket further comprises positioning the catheter hub between the first and second non-contiguous projections within the three-dimensional pocket.

24. The method of claim 23, further comprising moving a retaining strap of the catheter cover securement device from an open configuration allowing for insertion or removal of the catheter hub from the first and second non-contiguous projections to a closed configuration inhibiting insertion or removal of the catheter hub from the first and second non-contiguous projections.

25. The method of claim 24, wherein the first and second non-contiguous projections have a flat top surface, wherein the act of moving the retaining strap further comprises moving the retaining strap to the closed configuration sandwiching an engaged portion of the water-resistant sleeve in a flat condition against the flat top surface of the first and second non-contiguous projections.

26. The method of claim 25, wherein in the closed configuration, the three-dimensional pocket engages the catheter hub and a portion of the water-resistant sleeve, while the flat top surface supports an engaged portion of the water-resistant sleeve in the flat condition.

27. The method of claim 13, wherein the three-dimensional anchoring body is formed be from an elastomeric material.

28. The method of claim 13, wherein the rim circumferentially surrounds an exit point of the catheter through the skin, wherein the catheter cover securement device includes a curved perimeter edge configured to abut a portion of a water indicator.

29. A method of securing a catheter to a patient, wherein the catheter includes an exterior portion that extends outside of the patient, wherein the exterior portion of the catheter includes a catheter hub, the method comprising:
(a) securing a rim of a water-resistant sleeve to skin of the patient, wherein the catheter extends through the rim;
(b) after securing the rim of the water-resistant sleeve to the skin of the patient, securing a catheter cover securement device to the skin;
(c) after securing the rim of the water-resistant sleeve to the skin of the patient, securing the catheter hub of the catheter, which is surrounded by the water-resistant sleeve, within a three-dimensional pocket defined by a three-dimensional anchoring body of the catheter cover securement device while a retaining strap is in an open configuration; and
(d) after securing the catheter hub of the catheter within the three-dimensional pocket, moving the retaining strap to a closed configuration so that the retaining strap sandwiches an engaged portion of the water-resistant sleeve in a flat condition against a flat top surface of the three-dimensional anchoring body.

30. The method of claim 29, wherein the act of moving the retaining strap to the closed configuration further comprises moving the retaining strap to the closed configuration so that a longitudinal length of the flat top surface is equal to or greater than a maximum sleeve width.

31. A system for securing a catheter, the system comprising:
(a) a water-resistant sleeve defining a maximum sleeve width, wherein the water-resistant sleeve is configured to sheath the catheter; and
(b) a catheter cover securement device comprising:
(i) a three-dimensional anchoring body comprising:
(A) an inner side configured to be secured to skin of a patient,
(B) an outer side disposed opposite the inner side and configured to extend away from the skin,
(C) a top surface, wherein a longitudinal length of the top surface at least equal to the maximum sleeve width, and
(D) a three-dimensional pocket formed by the three-dimensional anchoring body, wherein the three-dimensional pocket is configured to receive a catheter hub of the catheter, and
(ii) a retaining strap movable relative to the top surface between open and closed configurations, wherein the retaining strap is configured to receive the catheter hub in the open configuration, wherein the retaining strap includes an engagement surface configured to conform to the top surface of the three-dimensional anchoring body to secure the catheter hub within the three-dimensional pocket in the closed configuration.

32. The system of claim 31, wherein the top surface of the anchoring body is a flat top surface, wherein the three-dimensional pocket communicates via a void with the flat top surface, wherein the flat top surface is continuous along the longitudinal length except for the void.
